(19) United States
(12) Patent Application Publication
(10) Pub. No.: US 2003/0021799 A1
(43) Pub. Date: Jan. 30, 2003

(54) Reverse transcriptase formulations

(76) Inventors: Jonathan Frome Wilkinson, Brighton (GB); Ariel Louwrier, Surrey (GB); Claire Louise Hunt, West Suffolk (GB)

Correspondence Address: Thomas M. Galgano, Esq., GALGANO & BURKE, Suite 135, 300 Rabro Drive, Hauppauge, NY, 11788, US

(21) Appl. No.: 10/193,454

(22) Filed: Jul. 10, 2002

Related U.S. Application Data
(60) Provisional application No. 60/304,463, filed on Jul. 11, 2001.

(51) Int. Cl. A61K 39/21; C12Q 1/70; C12Q 1/68; C12P 19/34; C12N 9/22

(52) U.S. Cl. 424/187.1; 435/6; 435/199; 435/91.2; 435/5

(57) ABSTRACT

This invention provides a novel formulation of reverse transcriptase enzymes comprising a reverse transcriptase derived from Avian Myeloblastosis Virus and combined with a reverse transcriptase derived from Moloney Murine Leukaemia Virus, and further provides an improved method of amplifying nucleic acid sequences by reverse transcription or coupled reverse transcription and polymerase chain reaction.

The formulation enhances sensitivity of RNA detection and increases product yield compared with either of the enzymes used separately.

### FIELD OF THE INVENTION

The present invention concerns improvements in and relating to reverse transcriptase formulations and includes a novel reverse transcriptase formulation and kit and uses for performing reverse transcription reactions.

### BACKGROUND TO THE INVENTION

Gene expression is a fundamental cellular process, in which DNA is transcribed to RNA, prior to protein translation. Gene expression differs from cell to cell, depending on many factors including the environment or state of differentiation of the cell, either with regard to the genes that are expressed, or the levels to which they are expressed. Consequently, measurement of the RNA status of a cell is key to understanding the normal physiological function of that cell, its interaction with other cells and its role in disease processes. Thus, gene expression is routinely analysed by laboratories working in a broad range of research areas.

A technique commonly used for the measurement of RNA in cells or tissues is reverse transcription (RT) and subsequent polymerase chain reaction (PCR) either in one reaction or using separate vessels for the RT and PCR stages. In this process, single-stranded RNA is reverse-transcribed to single-stranded cDNA by the reverse transcriptase (first strand synthesis). A second, complementary strand of DNA is then synthesised/replicated by a thermostable DNA polymerase, creating doubled-stranded cDNA (second strand synthesis). The double-stranded cDNA is subsequently amplified by PCR, whereby specific primer pairs complementary to the cDNA are extended by the DNA polymerase. The amplified DNA product can then be used for analysis and quantification.

Typically, RT and coupled RT-PCR are performed in suitable aqueous buffers that enable the reverse transcriptase and DNA polymerase to function. These buffered systems commonly comprise monovalent cations, such as sodium, potassium or ammonium; and/or magnesium ions, present in the concentration range 1-6 mM.

One particularly preferred biochemical buffer is Tris (hydroxymethyl) aminomethane hydrochloride, otherwise known as Tris hydrochloride, and which has a useful pH buffering range of between pH 7 and 9.

In addition to buffering agents the typical reverse transcription reaction mixture comprises: (a) deoxy nucleotide triphosphates as the building blocks to form the cDNA;

(b) the RNA template to be transcribed (either total RNA or mRNA) suitably present in the range 10 fg-1 μg; and (c) the reverse transcriptase enzyme.

For replication of the cDNA, (d) DNA polymerase and also (e) oligonucleotide primers complementary to the ‘sense’ (or ‘forward’) strand or to the ‘antisense’ (or ‘reverse’) strand are further provided.

Such a reaction mixture is suitably incubated at 37-60° C. for 5-60 minutes, to enable first strand synthesis to be catalysed by the reverse transcriptase. An aliquot of this mixture may then be removed and added to a separate PCR reaction. Whether a two stage separate vessel method is followed, or a coupled RT-PCR method, the reaction proceeds to the PCR stage, where DNA amplification is catalysed by the DNA polymerase.

PCR typically entails an initial incubation at 94-95° C. for 2 minutes to inactivate the reverse transcriptase and to denature the nucleotide strands. This is followed by 25-35 reaction cycles typically of:

This formulation is suitably adapted to be used in a reverse transcription and subsequent polymerase chain reaction and suitably can be used in a coupled reverse transcription and polymerase chain reaction performed in a single reaction vessel.

Preferably the formulation is packaged together with other reverse transcription reagents in a kit format. Such a kit is preferably adapted to enable the reactions to be performed in a single reaction vessel. The kit format formulation for use in a reverse transcription and polymerase chain reaction suitably has most or all of the reaction components for reverse transcription and polymerase chain reaction supplied in the kit.

Preferably the formulation includes a pH buffering agent to buffer the pH of the formulation, and particularly preferably the buffering agent buffers the pH of the formulation to a pH of 8 or greater and preferably about 8.8. This is notably higher than the normal pH optimum for MMuLV and, at pH 8.8 is even higher than the normal pH optimum for AMV.

Further in contrast to the normal reaction conditions for AMV or MMuLV, the formulation of the present invention operates best at temperatures of the order of 47° C. being significantly above the MMuLV optimal range.

The ratio of units of enzyme of Moloney Murine Leukaemia Virus to Myeloblastosis Virus is balanced toward Moloney Murine Leukaemia Virus, preferably being between 3:2 and 7:1 and preferably about 4:1.

The present invention also provides for the use of a formulation as defined above in a reverse transcription reaction and which is suitably further in combination with a polymerase chain reaction.

The use preferably entails the aforementioned reaction conditions of pH greater than 8 and temperature above 42° C. and peferably of the order of 47° C.

The invention further provides a kit for use in a reverse transcription reaction and comprising a reverse transcriptase derived from Moloney Murine Leukaemia Virus and a reverse transcriptase derived from Avian Myeloblastosis Virus together with instructions to combine the reverse transcriptases to form a formulation as defined above.

An unexpected and valuable consequence of this novel combined transcriptase formulation, as applied to RT or RT-PCR, is a very significant improvement in sensitivity and product yield over either of the enzymes used separately, for standardised conditions.

### BRIEF DESCRIPTIONS OF THE DRAWINGS

Preferred embodiments of the present invention will now be described by way of experimental examples with reference to the accompanying figures in which:

FIGS. 1A to 1E comprise photographs of an agarose gel electrophoresis result from RT-PCR for each of five different reaction mixtures (a) to (e) in accordance with a first experimental example; and

FIGS. 2A to 2E comprise photographs of an agarose gel electrophoresis result from RT-PCR for each of five further different reaction mixtures (a) to (e) in accordance with a second experimental example.

### DESCRIPTION OF PREFERRED EMBODIMENTS

### EXAMPLE 1

Coupled RT-PCR was performed to illustrate the increased sensitivity of the reverse transcriptase blend, compared to either AMV or MMuLV used separately.

MS2 RNA (Boehringer Mannheim) was serially diluted in 10-fold steps from 1 ng to 1 fg. Each of these were added to a separate respective reaction mixture containing, in a 50 μl volume:

1.25 Units of Taq DNA polymerase

75 mM Tris HCl buffer (pH 8.8)

20 mM NH₄(SO₄)

0.02% (v/v) Tween 20 solvating agent

0.2 mM each of dATP, dCTP, dGTP and dTTP deoxynucleotides

10 pmol of MS2 primer 1 (5′ CTAACGGGGTCGCTGMT 3′)

10 pmol of MS2 primer 2 (5′ AGGTCGGATGCTTTGTGA 3′) and either (a) 0.1 Units AMV, (b) 0.5 Units AMV, (c) 0.4 Units MMuLV, (d) 0.5 Units MMuLV or (e) 0.1+0.4 Units of the AMV+MMuLV blend reverse transcriptases.

The reaction mixtures were then subjected to RT-PCR under the following conditions:

47° C. for 30 minutes

94° C. for 2 minutes

40 cycles of

94° C. for 20 seconds

60° C. for 30 seconds

72° C. for 40 seconds

72° C. for 5 minutes.

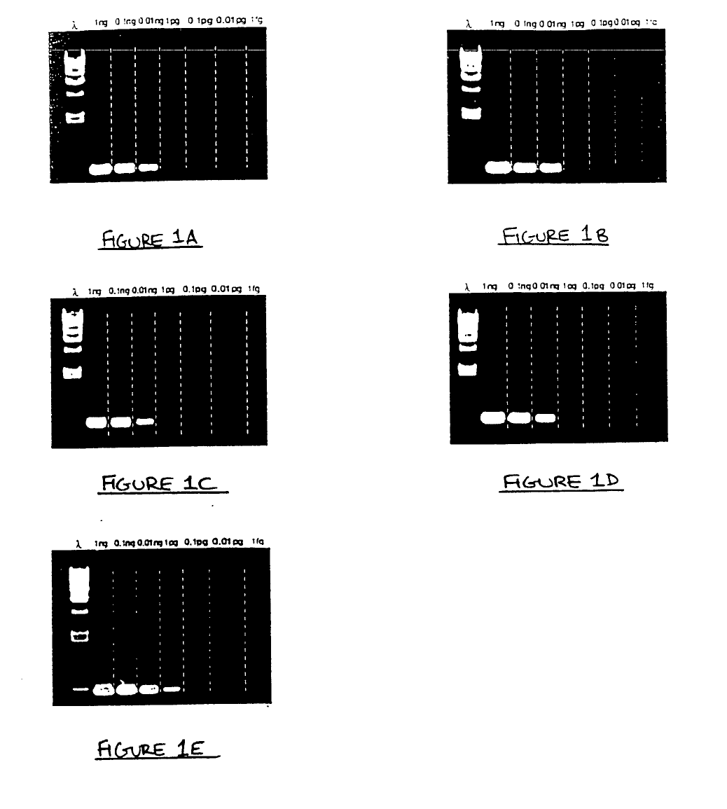

The products were separated by TAE agarose gel electrophoresis on a 1% (w/v) agarose gel alongside a X Hind III marker for comparison (sizes 23130, 9416, 6557, 4361, 2322, 2027, 564 and 125 base pairs. The gel was stained with ethidium bromide, and DNA products visualised using a UV transiluminator (FIG. 1). RTPCR using the MS2 template and primers generates a product of 503 bp as can be seen for each of the reaction mixtures (a) to (e) (FIGS. 1A-E). Additionally, it should be noted that MS2 product is generated from template levels as low as 1 pg for each of the reverse transcriptases separately, at either concentration used (FIGS. 1A-D). However, a much greater sensitivity was achieved with the blend of reverse transcriptases, detectable product being amplified from as little as 10 fg of RNA template (FIG. 1E).

### EXAMPLE 2

Coupled RT-PCR was again performed to illustrate the increased product yield achieved using the reverse transcriptase blend, compared to either AMV or MMuLV used separately but this time using testicular total RNA. The testicular total RNA (Clontech) was serially diluted in 10-fold steps from 0.1 μg to 10 pg. Each dilution was added to a reaction mixture containing, in a 50 μl volume:

1.25 Units of Taq DNA polymerase

75 mM Tris HCl (pH 8.8)

20 mM NH₄(SO₄)

0.02% (v/v) Tween 20

0.2 mM each of dATP, dCTP, dGTP and dTTP

25 pmol β-Actin Primer 1 (5′ AGAGATGGCCACGGCTGCTT 3′)

25 pmol β-Actin Primer 2 (5′ ATTTGCGGTGGACGATGGAG 3′) and either (a) 0.1 Units AMV, (b) 0.5 Units AMV, (c) 0.4 Units MMuLV, (d) 0.5 Units MMuLV or (e) 0.1+0.4 Units of the AMV+MMuLV blend.

The reaction mixtures were then subjected to RT-PCR under the following conditions:

47° C. for 30 minutes

94° C. for 2 minutes

40 cycles of

94° C. for 20 seconds

60° C. for 30 seconds

72° C. for 40 seconds

72° C. for 5 minutes.

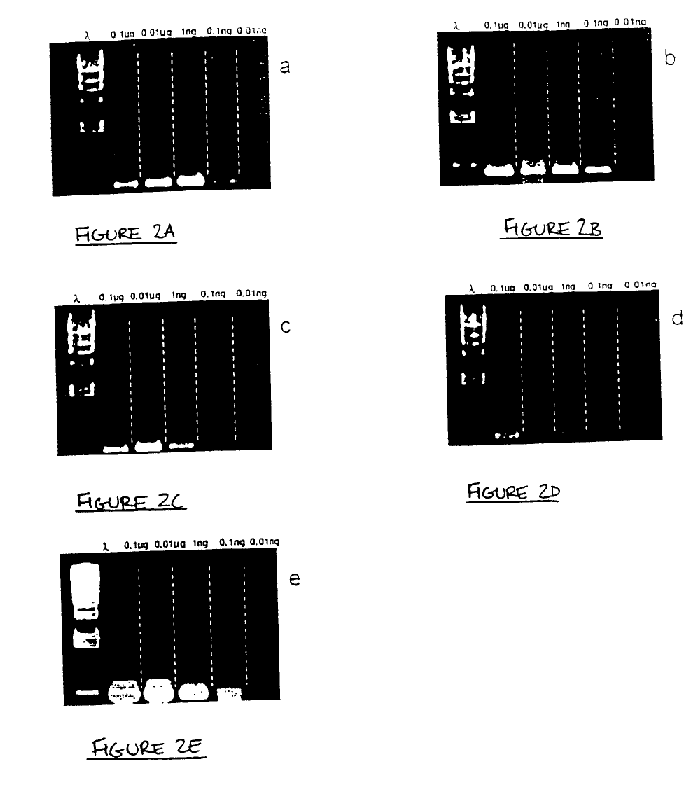

The products were separated by TAE agarose gel electrophoresis on a 1% (w/v) agarose gel, the gel stained with ethidium bromide, and DNA products visualised using a UV transiluminator (FIG. 2). RT-PCR using β-actin primers and total RNA generated a DNA product of 426 bp as can be seen for each of the reaction mixtures (a) to (e) as shown in FIGS. 2(A-E).

Product was detectable across the range of RNA template concentrations used (0.1 μg to 10 pg), with 0.5U of AMV (FIG. 2B) and with the blend of AMV+MMuLV (FIG. 2E). However the product yield across this RNA template range was much greater for the blend of reverse transcriptases, than for either of the reverse transcriptases used separately, at either concentration.

From the above it is readily apparent that the combination of AMV and MMuLV provides greater sensitivity and product yield when present in the same overall amounts, than either of the enzymes used alone.

1. A formulation having reverse transcriptase activity and comprising a combination of a reverse transcriptase derived from Moloney Murine Leukaemia Virus and a reverse transcriptase derived from Avian Myeloblastosis Virus.

2. A formulation as in claim 1, that can be used in a reverse transcription and subsequent polymerase chain reaction.

3. A formulation as in claim 2, that can be used in a coupled reverse transcription and polymerase chain reaction performed in a single reaction vessel.

4. A formulation as claimed in claim 1, wherein the formulation is packaged together with other reverse transcription reagents in a kit format.

5. A formulation as claimed in claim 4 wherein the kit is adapted to enable the reactions to be performed in a single reaction vessel.

6. A formulation as claimed in claim 4, that can be used in a reverse transcription and polymerase chain reaction wherein most or all of the reaction components for reverse transcription and polymerase chain reaction are supplied in the kit.

7. A formulation as claimed in claim 1 wherein the formulation includes a pH buffering agent to buffer the pH of the formulation.

8. A formulation as claimed in claim 7 wherein the buffering agent buffers the pH of the formulation to a pH of 8 or greater and preferably about 8.8.

9. A formulation as claimed in claim 1 wherein the ratio of units of enzyme of Moloney Murine Leukaemia Virus to Myeloblastosis Virus is balanced toward Moloney Murine Leukaemia Virus, preferably being between 3:2 and 7:1 and preferably about 4:1.

10. Use of a formulation as claimed in claim 1 in a reverse transcription reaction.

11. Use as claimed in claim 10 further in combination with a polymerase chain reaction.

12. Use as claimed in claim 10, wherein the pH of the reaction mixture is greater than 8 and/or the temperature of the reaction mixture is greater than 42° C. and preferably about 47° C.

13. A kit for use in a reverse transcription reaction and comprising a reverse transcriptase derived from Moloney Murine Leukaemia Virus and a reverse transcriptase derived from Avian Myeloblastosis Virus together with instructions to combine the reverse transcriptases to form a formulation as claimed in 1.
